(19) United States
(12) Patent Application Publication
(10) Pub. No.: US 2023/0382836 A1
(43) Pub. Date: Nov. 30, 2023

(54) AGRICULTURAL PRODUCTS FOAM CONTROL AGENT

(71) Applicants: Dow Global Technologies LLC, Midland, MI (US); Dow Silicones Corporation, Midland, MI (US)

(72) Inventors: Timothy A. Roggow, II, Chesaning, MI (US); Xue Chen, Manvel, TX (US); Haseeb Moten, Midland, MI (US)

(21) Appl. No.: 18/248,879

(22) PCT Filed: Oct. 21, 2021
(86) PCT No.: PCT/US2021/055936

§ 371 Date: Apr. 13, 2023

Related U.S. Application Data
(60) Provisional application No. 63/105,389, filed on Oct. 26, 2020.

(51) Int. Cl. C07C 31/125; C05G 3/50; A01N 25/30

(52) U.S. Cl. CPC C07C 31/125; C05G 3/50; A01N 25/30

(57) ABSTRACT

A foam control agent and method of controlling foam for agricultural products by use of a foam control agent, wherein the agent comprises at least a branched alcohol.

Embodiments relate to a foam control agent and method of controlling foam in agricultural products, wherein the agent comprises at least a branched alcohol.

### INTRODUCTION

Agricultural products such as pesticides, herbicides, micronutrients, and fungicides are commonly found in liquid form. Whether these products are manufactured, packaged, used, or applied as a liquid, the formation of foam is a common issue. This is because agricultural liquids commonly contain components such as surfactants, dispersants, rheology modifiers, and solids that stabilize air/water interfaces. Stabilization of this air/water interface can result in the formation of foam.

Foam formation presents challenges in the production, packaging, transport, and application of liquid agricultural products. Unintended worker and environmental exposure, inability to fill packaging, inability to pump the liquid, and uneven application rates are examples of the challenges that the presence of foam can present.

For all these reasons and more, there is a need for a foam control agent and method of controlling foam in agricultural products.

### SUMMARY

Embodiments relate to a foam control agent and method of controlling foam for agricultural products, wherein the agent comprises at least a branched alcohol. This organic defoamer can also boost performance of silicone defoamers.

### DETAILED DESCRIPTION

The present disclosure relates to a foam control agent for agricultural products. The present disclosure details how, unexpectedly, branched alcohols have been shown to have superior foam control performance. The branched alcohols may be 2-alkyl-1-alkanols (also known as Guerbet alcohols), and preferably 2-ethylhexanol (2-EH) and 2-propylheptanol (2-PH). These alcohols can be synthesized via the aldol condensation of the corresponding aldehydes or from the Guerbet reaction of primary linear alcohols. Other methods of production may also be utilized.

In this invention, C8 to C12 β-branched alcohols (C9-C12 Guerbet alcohols) were found to be surprisingly effective in reducing the foam during the various stages of agricultural products. Another benefit to the branched alcohols is their very good biodegradability.

The generic structure of the antifoaming agent currently disclosed is as follows:

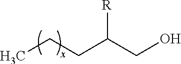

wherein x is an integer from 2 to 8 and R is an alkyl group with 1-8 carbon atoms.

The foam control agent may also be described as comprising a 2-alkyl substituted alcohol from C8-C12. The alcohols can be predominately one isomer (>95 wt. %) or a mixture of alcohols which can be generated by an aldol condensation of a mixture of aldehydes or generated from a mixture of alcohols via the Guerbet reaction.

The C8-C32 Guerbet alcohols including 2-ethylhexanol, 2-butyl-1-octanol, and 2-propylheptanol and the mixture of C8, C9, and C10 alcohols generated from the aldol condensation of butyraldehyde and valeraldehyde are preferred in some embodiments.

The concentration of the Guerbet alcohol in the formulated foam control agent ranges from 0.01% to 100%, preferably ranging from 30% to 100% when used as antifoaming agent or as a defoaming agent. The Guerbet alcohol can be in the form of a solid or liquid, a liquid is preferred. If it is a solid, the material may be dissolved or dispersed in a solvent. The said foam control agent can be aqueous solution or organic solvent-based solution.

Other foam control agents (e.g., copolymers composed of ethylene oxide, propylene oxide, and/or butylene oxide, random or blocks) or other hydrophobic materials such as waxes, oils or silicas may also be added with the branched, Guerbet alcohol(s). Silicone defoamers can be used in conjunction with the 2-alkyl alcohols. Surfactants, especially alkoxylates of the alcohols can also be used. The use of branched alcohols as foam control agents may be water based or oil based.

The new foam control agent presently disclosed may be in the form of a solid or liquid. If it is a solid, the material may be dissolved or dispersed in a solvent before use as a foam control agent. The presently disclosed agents are believed to work in the presence of all commonly used agricultural products.

The chemical agent can be used both in antifoamer or defoamer formulations. Antifoamer formulations are obtained by the mixture of polyglycols, esters, silicones, solvents, water and other chemicals that in the gas-liquid interface of the bubble avoiding the foam formation. Other amphiphilic chemicals based on block copolymer can be used as well. In defoaming formulations, in addition to the products mentioned above, it can be used vegetal oils, mineral oils, waxes and other oily agents.

The optional surfactant or emulsifier contained in the foam control agent is selected to be suitable for improving the compatibility of the foam control agent on the feedstock or forming an emulsion with the composition of branched alcohol. The optional surfactant or emulsifier has an amount ranging from 0.1-30% by weight of the composition of branched alcohol.

The optional surfactant or emulsifier may be anionic, cationic or nonioic. Examples of suitable anionic surfactants or emulsifiers are alkali metal, ammonium and amine soaps; the fatty acid part of such soaps contains preferably at least 10 carbon atoms. The soaps can also be formed “in situ;” in other words, a fatty acid can be added to the oil phase and an alkaline material to the aqueous phase.

Other examples of suitable anionic surfactants or emulsifiers are alkali metal salts of alkyl-aryl sulfonic acids, sodium dialkyl sulfosuccinate, sulfated or sulfonated oils, e.g., sulfated castor oil; sulfonated tallow, and alkali salts of short chain petroleum sulfonic acids.

Suitable cationic surfactants or emulsifiers are salts of long chain primary, secondary or tertiary amines, such as oleylamide acetate, cetylamine acetate, di-dodecylamine lactate, the acetate of aminoethyl-aminoethyl stearamide, dilauroyl triethylene tetramine diacetate, 1-aminoethyl-2-heptadecenyl imidazoline acetate; and quaternary salts, such as cetylpyridinium bromide, hexadecyl ethyl morpholinium chloride, and diethyl di-dodecyl ammonium chloride.

Examples of suitable nonionic surfactants or emulsifiers are condensation products of higher fatty alcohols with ethylene oxide, such as the reaction product of oleyl alcohol with 10 ethylene oxide units; condensation products of alkylphenols with ethylene oxide, such as the reaction product of isoctylphenol with 12 ethylene oxide units; condensation products of higher fatty acid amides with 5, or more, ethylene oxide units; polyethylene glycol esters of long chain fatty acids, such as tetraethylene glycol monopalmitate, hexaethyleneglycol monolaurate, nonaethyleneglycol monostearate, nonaethyleneglycol dioleate, tridecaethyleneglycol monoarachidate, tricosaethyleneglycol monobehenate, tricosaethyleneglycol dibehenate, polyhydric alcohol partial higher fatty acid esters such as sorbitan tristearate, ethylene oxide condensation products of polyhydric alcohol partial higher fatty acid esters, and their inner anhydrides (mannitol-anhydride, called Mannitan, and sorbitol-anhydride, called Sorbitan), such as glycerol monopalmitate reacted with 10 molecules of ethylene oxide, pentaerythritol monooleate reacted with 12 molecules of ethylene oxide, sorbitan monostearate reacted with 10-15 molecules of ethylene oxide, mannitan monopalmitate reacted with 10-15 molecules of ethylene oxide; long chain polyglycols in which one hydroxyl group is esterified with a higher fatty acid and other hydroxyl group is etherified with a low molecular alcohol, such as methoxypolyethylene glycol 550 monostearate (550 meaning the average molecular weight of the polyglycol ether). A combination of two or more of these surfactants may be used; e.g., a cationic may be blended with a nonionic or an anionic with a nonionic.

The foam control agent may further comprise one or more additives. Examples of additives include ethylene oxide/propylene oxide block copolymers, butylene oxide/propylene oxide block copolymers, ethylene oxide/butylene oxide block copolymers, waxes, or silicone-based materials. For other agricultural applications where surfactants cause foaming in products, higher 2-alkyl substituted alcohols up to C32 can be used.

The Guerbet alcohol, or formulation thereof, may be present as a solution, suspension, emulsion, dispersion, or dry product. The foam control agent (containing Guerbet alcohol) described wherein may be utilized in, but not limited to agricultural applications such as fertilizers, micronutrients or pesticides. The types of formulation with which this agent may be utilized includes, but not limited to: Suspension Concentrates (SC), Emulsion Concentrates (EC), Capsule Suspensions (CS), Micro-emulsions (ME), Emulsions: oil in water (EW). Suspo-emulsions (SE), Soluble Concentrates (SL), Water Dispersible Granules (WG), Water Soluble Powders (SP), Wettable Powders (WP), and Oil Dispersions (OD).

### Examples

An experiment to test the efficacy of the presently disclosed foam control agent and others may be conducted as follows.

Materials

TABLE 1
Raw Materials

| | Producer/ | | Chemistry |
|---|---|---|---|
| Name | Vendor | Function | and function |
| Big N' Tough | Tractor Supply | Foam medium | Commercial |
| (41% glyphosate | Company | | Glyphosate |
| concentrate) | | | salt solution |
| 2-Propylheptanol | Sigma Aldrich | Tested | |
| (2-PH) | | Example | |
| 2-Ethylhexanol | Sigma Aldrich | Tested | |
| (2-EH) | | Example | |
| Xiameter ACP- | Dow Chemical | Comparative | Silicone based |
| 1400 Antifoam | | Example | foam control agent |
| Compound | | | |
| Propylene Glycol | Sigma Aldrich | Diluent for silicone compounds | 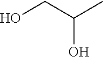 |

TABLE 2
Test Formulations

| | | | Actives | Foaming |
|---|---|---|---|---|
| Examples | Foam control agent | Amount | Concentration | Medium |
| Example 1 | 2-Propylheptanol | 2.5 grams of a | 50 ppm | 2.5% Big N′ |
| | | 2000 ppm | | Tough |
| | | dilution of 2- | | Glyphosate |
| | | propyl heptanol | | solution |
| | | propylene | | |
| | | glycol | | |
| Example 2 | ACP-1400 + 2- | 2.5 grams of a | 1 ppm + 50 ppm | 2.5% Big N′ |
| | Propylheptanol | 2000 ppm | | Tough |
| | | dilution of 2- | | Glyphosate |
| | | propyl heptanol | | solution |
| | | propylene | | |
| | | glycol and 0.5 | | |
| | | grams of | | |
| | | 200 ppm dilution | | |
| | | of ACP-1400 in | | |
| | | propylene | | |
| | | glycol | | |
| Example 3 | ACP-1400 at + 2- | 2.5 grams of a | 10 ppm + 50 ppm | 2.5% Big N′ |
| | Propylheptanol | 2000 ppm | | Tough |
| | | dilution of 2- | | Glyphosate |
| | | propylheptanol | | solution |
| | | propylene | | |
| | | glycol and 0.5 | | |
| | | grams of | | |
| | | 200 ppm 2- | | |
| | | propyl heptanol. | | |
| Example 4 | 2-Propylheptanol | 0.1 grams of 2- | 1,000 ppm | 1% Triton |
| | | Propylheptanol | | X-100 |
| Example 5 | ACP-1400 + 2-Propyl | 0.5 grams of a | 20 ppm + 1000 ppm | 1% Triton |
| | Heptanol | 4,000 ppm | | X-100 |
| | | dilution of | | |
| | | ACP-1400 | | |
| | | compound in | | |
| | | propylene | | |
| | | glycol, and | | |
| | | 0.1 grams of | | |
| | | 0.1 grams of 2- | | |
| | | propyl heptanol | | |
| Example 6 | 2-Ethylhexanol | 0.5 grams of 2- | 5,000 ppm | 1% Triton |
| | | Ethylhexanol | | X-100 |
| Example 7 | ACP-1400 + 2- | 0.1 grams of 2- | 50 ppm + 1,000 ppm | 1% Triton |
| | Ethylhexanol | ethyl hexanol, | | X-100 |
| | | and 0.5 grams | | |
| | | of a 10,000 ppm | | |
| | | dilution of | | |
| | | ACP-1400 in | | |
| | | propylene | | |
| | | glycol | | |
| Comparative | ACP-1400 | 0.5 grams of a | 1 ppm | 2.5% Big N′ |
| Example 1 | | 200 ppm dilution | | Tough |
| | | of ACP-1400 | | Glyphosate |
| | | compound in | | solution |
| | | propylene | | |
| | | glycol | | |
| Comparative | ACP-1400 | 0.5 grams of a | 10 ppm | 2.5% Big N′ |
| Example 2 | | 2,000 ppm | | Tough |
| | | dilution of | | Glyphosate |
| | | ACP-1400 | | solution |
| | | compound in | | |
| | | propylene | | |
| | | glycol | | |
| Comparative | ACP-1400 | 0.5 grams of a | 20 ppm | 1% Triton |
| Example 3 | | 4,000 ppm | | X-100 |
| | | dilution of | | |
| | | ACP-1400 | | |
| | | compound in | | |
| | | propylene | | |
| | | glycol | | |
| Comparative | ACP-1400 | 0.5 grams of a | 50 ppm | 1% Triton |
| Example 4 | | 10,000 ppm | | X-100 |
| | | dilution of | | |
| | | ACP-1400 | | |
| | | compound in | | |
| | | propylene | | |
| | | glycol | | |

Note: Dilutions in propylene glycol were completed by weighing the appropriate weights of propylene glycol and ACP-1400 to make a 50-gram batch into a 4-ounce glass jar. The jar was capped and shaken vigorously by hand for 30 seconds to mix.

Testing Methodology

A shake test was conducted on the various examples listed above. The equipment utilized for said test was a Burrell WRIST-ACTION Model AA shaker, equipped with a suitable clamp to accommodate an 8 oz (240 mL) French Square bottle (Burrell Corp., Pittsburgh, PA, Cat. No. 75-755-04). The shaker arm measured 5¼+/− 1/16 in. (13.34+/−0.16 cm). A modified method may use a shaker arm that measures 9 in.+/−1.4 in. This length is measured from the center of the shaker shaft to the center of the bottle. It should be noted a longer shaft length tends to produce more foam, thus is a more challenging test. The arm should be horizontal in the rest position to hold the bottle in a vertical position. The shaking arc should be around 16 degrees and the frequency around 350 strokes per minute.

For Comparative Examples 1-2 and Examples 1-3 the shake test was conducted wherein the shaker arm was affixed at a radius of 9 in. as measured from the center of the shaker shaft to the center of the bottle. The arm was horizontal in the rest position to hold the bottle in a vertical position. The shaking arc was approximately 16 degrees and the frequency approximately 350 strokes per minute.

Each formulation was shaken four cycles consisting of 8, 32, 48, and 96 second successive durations. For each duration of time it took for the foam to collapse was recorded and for the foam to break. Collapse is defined as the time for the foam to fall to less than 0.5 cm over most of the surface of the given tested liquid when shaking was stopped. Time for the foam to break was defined as the time for a clear surface to appear upon the surface of the test liquids through the foam once shaking is stopped.

Comparative Examples 3-4 and Examples 4-7 were also tested using a shake test. For this test, the shaker arm was approximately 5 in. measured from the center of the shaker shaft to the center of the bottle. The arm was horizontal in the rest position to hold the bottle in a vertical position. The shaking arc was approximately 16 degrees and the frequency approximately 350 strokes per minute. Each formulation was shaken for 30 seconds. After shaking stopped the time was recorded for the foam to collapse. Each formulation was subjected to 14 cycles of shaking and foam collapse. If a sample failed to collapse in less than 300 seconds the testing was discontinued. Foam collapse was defined as the foam height falling to less than 0.5 cm over most of the surface.

Results Foam control performance for the foam control agents are shown below in Tables 3-5.

TABLE 3
Foam Collapse in Glyphosate Solution

| Shake | Comparative | Comparative | Example | Example | Example |
|---|---|---|---|---|---|
| Time (S) | #1 | #2 | #1 | #2 | #3 |
| 8 | 23 | 2.19 | 9.20 | 19.43 | 7.15 |
| 32 | 115 | 8.03 | 42.04 | 26.58 | 9.11 |
| 48 | 180 | 10.13 | 48.70 | 26.25 | 9.81 |
| 96 | 180 | 19.66 | 59.83 | 32.88 | 16.75 |

TABLE 4
Foam Break in Glyphosate Solution

| Shake | Comparative | Comparative | Example | Example | Example |
|---|---|---|---|---|---|
| Time (S) | #1 | #2 | #1 | #2 | #3 |
| 8 | 180 | 9.3 | 39.8 | 35.78 | 10.87 |
| 32 | 180 | 180 | 42.08 | 32.62 | 14.25 |
| 48 | 180 | 180 | 56.82 | 32.35 | 15.63 |
| 96 | 180 | 180 | 67.37 | 39.23 | 24.1 |

TABLE 5
Foam Collapse in 1% Triton X-100

| | | | | | Comparative | Comparative |
|---|---|---|---|---|---|---|
| | Example 4 | Example 5 | Example 6 | Example 7 | Example 3 | Example 4 |
| Detail | 1000PPM | 20PPM ACP | 5000PPM | 50PPM ACP | 20PPM ACP 1400 | 50PPM ACP |
| | 2PH | 1400 + 1000 | EH | 1400 + 1000 | | 1400 |
| | | PPM 2PH | | PPM EH | | |
| Cycle | Collapse | Collapse | Collapse | Collapse | Collapse Time (s) | Collapse Time |
| | Time (s) | Time (s) | Time (s) | Time (s) | | (s) |
| 1 | 23.57 | 7.94 | 6.95 | 3.84 | 17.39 | 15.03 |
| 2 | 300 | 22.64 | 300 | 17.72 | 29.61 | 20.98 |
| 3 | | 30.05 | | 22.81 | 41.16 | 21.88 |
| 4 | | 40.82 | | 31.16 | 66.11 | 31.3 |
| 5 | | 35.51 | | 23.65 | 76.76 | 37.19 |
| 6 | | 33.64 | | 29.46 | 132.89 | 52.21 |
| 7 | | 32.64 | | 36.40 | 300 | 73.74 |
| 8 | | 39.61 | | 36.16 | | 190.23 |
| 9 | | 42.41 | | 41.34 | | 300 |
| 10 | | 39.02 | | 48.34 | | |
| 11 | | 46.19 | | 53.85 | | |
| 12 | | 58.54 | | 65.87 | | |
| 13 | | 73.08 | | 85.14 | | |
| 14 | | 111.24 | | 300 | | |

As shown in Tables 3-5, Example 1 (2-propyl heptanol) surprisingly provides effective foam control as the sole defoamer in an agricultural formulation. Examples 2 and 3 also indicate surprising synergistic performance of the 2-propyl heptanol in combination with the silicone compound additive which can improve the collapse times for extended shear conditions and is especially effective in improving the foam break performance in an agricultural formulation.

Example 4 indicates that 2-propyl heptanol provides foam control as the sole defoamer in a non-ionic surfactant solution, a surfactant system common to agricultural applications. Example 5 indicates that 2-ethyl hexanol also provides foam control as the sole defoamer in a non-ionic surfactant solution, a surfactant system common to agricultural applications. Example 6 indicates that the 2-propyl heptanol in combination with a silicone compound additive can improve the foam control performance in a non-ionic surfactant solution, a surfactant system common to agricultural applications. Example 7 indicates that the 2-ethyl hexanol in combination with a silicone compound additive can improve the foam control performance in a non-ionic surfactant solution, a surfactant system common to agricultural applications. All of these are surprising and helpful results and show clearly superior performance to the Comparative Examples tested.

1. (canceled)

2. (canceled)

3. (canceled)

4. (canceled)

5. A method of controlling foam for agricultural products by use of a foam control agent, wherein the agent comprises at least a branched alcohol that has the structure of: 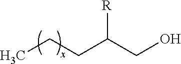
wherein x is an integer from 2 to 8 and R is an alkyl group with 1-8 carbon atoms, wherein the alcohol has from 8 to 12 carbon atoms.

6. The method of claim 5, wherein at least one other foam control agent or hydrophobic material is added.

7. The method of claim 5, wherein silicone is also added.

8. The method of claim 5, wherein the method is used for agricultural products.

9. The method of claim 8, wherein the agricultural products are liquid fertilizers, micronutrients or pesticides.
